(19) United States
(12) Patent Application Publication
(10) Pub. No.: US 2004/0126883 A1
(43) Pub. Date: Jul. 1, 2004

(54) Method for producing a multi-gene recombinant vector construct and the application

(76) Inventor: Yaoguang Liu, Guangzhou (CN)

Correspondence Address: ROSENBERG, KLEIN & LEE, 3458 ELLICOTT CENTER DRIVE-SUITE 101, ELLICOTT CITY, MD, 21043, US

(21) Appl. No.: 10/658,286

(22) Filed: Sep. 10, 2003

(30) Foreign Application Priority Data
Sep. 30, 2002 (CN) 02134869.3

(51) Int. Cl. C12P 19/34; C12N 15/85

(52) U.S. Cl. 435/455; 435/320.1; 435/91.2

(57) ABSTRACT

The present invention provides a method and a multi-gene assembly vector system having an acceptor vector and two donor vectors for effective transfer of multiple genes into a single vector. Using the method and the vector system disclosed in this invention, multiple cycles of DNA recombination based on a recombination system can be repeatedly carried out by alternate use of the two donor vectors together with the acceptor vector for recombination. The vector system and the method are used to construct any genetic engineering vector that contains multiple genes or DNA fragments of interest for transfer and expression of the genes in different cells, tissues, and organisms.

### BACKGROUND OF THE INVENTION

1. Field of the Invention

The present invention is related to the biotechnology field, in particular a method and vectors for producing multi-gene recombinant DNA and their applications in biotechnology.

2. Description of Related Art

Genetic transformation is a basic technology in genetic engineering and is used to introduce genes into cells of an organism. The majority of experiments and applications performed to date involve the manipulation of a single or a few genes. However, many important traits and complex metabolic pathways depend upon interactions among a number of genes. Therefore, attempts have been made to introduce multiple genes into cells of an organism to manipulate polygenic traits and multiple traits, and produce multiple gene products. However, genetic transformation with multiple genes is encumbered by technical limitations of current technologies.

At present, the techniques used for introduction of multiple genes into organisms include:

(1) co-transformation with mixed multiple plasmid vectors containing different genes using particle bombardment and other methods (Chen et al., 1998; Ye et al, 2000);

(2) sequential re-transformation of the same recipient organism with vectors where each vector contains one or a few genes (Lapierre et al, 1999); or sexual crossing between transgenic organisms carrying different transgenes to recombine the genes to a single organism (Ma and Hiatt, 1995); and

(3) linking of multiple genes of different sources into the same vector using conventional molecular cloning technology for transformation (Van Engelen et al, 1994; Daniell et al, 2001).

Although the first technique (1) is simple, the efficiency of co-transformation with multiple plasmids decreases progressively with the increase of the number of plasmids. Furthermore, co-transformation with separate plasmids is a random event, thus the insertion copy numbers and the relative arrangement among transgenes cannot be controlled. Therefore, some genes may insert into the genome, and some genes may not.

For the second technique (2), the selectable marker for transformation must be removed from the transgenic organisms, or a different selectable marker must be used in each new round of transformation before the next round of transformation. In addition, multiple rounds of transformation or cross between transgenic organisms are very time-consuming, and hence this technique is rarely used.

The third technique (3) is the most commonly used and reliable approach. However, only a small number of genes can be cloned into a single vector with the present molecular cloning technology (Halpin et al, 2001) where the introduction of multiple genes into a single vector is limited, for example, to no more than four or five genes. This limitation has three main aspects. (1) When sequentially combining multiple foreign DNA sequences into a vector, the size of the recombinant vector will increase accordingly. Consequently, unique cloning sites that usually are restriction endonuclease cutting sites will decrease, and finally no suitable cloning sites will be available. (2) When the size of the recombinant vector increases, the ligation efficiency between a new DNA fragment and the vector decreases, especially for fragments with blunt ends. (3) The cloning capacity of the conventional plasmid vectors (for example the pUC vectors and its derivatives) is low so cloning of multiple genes using these vectors is difficult. Although the cloning capacity of some vectors such as those based on an F-factor or P1 replicon, e.g. bacterial artificial chromosomes (BAC, P1), a binary bacterial artificial chromosome (BIBAC) and a transformation-competent artificial chromosome (TAC), is larger than others (Stemberg et al., 1990; Shizuya et al., 1992; Halmilton, 1997; Liu et al., 1999), the vectors with large cloning capacities are only suitable for cloning a single large DNA fragment but not for cloning multiple DNA fragments from different sources.

DNA recombination is defined as the exchange of DNA molecules catalyzed by recombination enzymes (recombinases). DNA recombination mediated by recombinase is a continuous process of cleavage, exchange and re-ligation of DNA strands. Several DNA recombination systems, including homologous recombination and site-specific recombination systems, have been discovered. Recombination systems such as Cre/loxP, Rlp/FRT, R/Rs, attB/attP and Gin/Gix systems enable recombination to occur between two specific recombination sites and thus can be used for gene integration or gene removal (Stemberg et al, 1981; Nash, 1981; Mcleld et al, 1986; Merker et al, 1993). For example, the Cre recombinase catalyzes recombination between two plasmids to produce a single recombinant (integrative) plasmid where each plasmid has a loxP recombination site (34 base composed). Reverse recombination also occurs between two loxP sites in directed-orientation in the integrative plasmid to produce two separate plasmids. Although these recombination systems have been successfully used in the recombination of target genes, current methodologies only allow one or two rounds of recombination (McCormac et al.,1 999), and no effective methods are available for multiple rounds of gene recombination to insert multiple genes into a single vector.

Accordingly, effective methods to insert multiple genes of interest into a single vector are needed for manipulation of multiple genes for either applied or academic purposes.

### SUMMARY OF THE INVENTION

The present invention provides a method for effective assembly of multiple genes or DNA fragments into single genetic engineering vectors and a vector system employed for such purpose. The invention includes but is not limited to: (i) a multi-gene assembly vector system comprising an acceptor vector and at least two donor vectors; (ii) a method comprising a DNA recombination system allowing multiple rounds of gene assembly by sequential DNA delivery into an acceptor vector via DNA swapping between the acceptor vector and different donor vectors; and (iii) specially designed DNA sequences including cutting sites for rare-cutting endonucleases and irreversible recombination sites on the acceptor and donor vectors for removing backbone fragments of the donor vector from the integrative plasmid intermediate during each round of recombination.

The method in accordance with the present invention allow the manipulation of multiple genes in genetic engineering and the study of gene functions including but not limited to transfer or expression of multiple genes into recipients including but not limited to cells, tissues and organisms. Examples are given in the present invention to demonstrate the capability and effectiveness of the method to synthesize a single recombinant plasmid vector carrying a large number of genes and DNA fragments, and of the subsequent transfer of the linked genes and DNA fragments into the rice genome.

In the present invention, individual components including a DNA recombination system, homing endonuclease cutting sites, the TAC and other vector elements, which are currently used for other purposes, are compiled into a new vector system to create a novel technology for link of multiple genes in a single vector by multiple rounds of gene recombination. The present invention overcomes the technical limitations experienced with existing methods for synthesis of multi-gene vector constructs. The present invention is not limited by the nature of the recombinase target site for recombination employed. In one embodiment, the recombinase target site can be selected from the group consisting of lox, FRT, Rs, att, Gix, or their mutant sites. The present invention is not limited by the nature of the rare-cutting sites or irreversible recombination sites employed either. In one embodiment, the sites can be selected from the group consisting of homing endonuclease sites I-Sce I, I-Ceul, I-PpoI, I-TliI, PI-SceI (VDE) or PI-PspI or of irreversible specific recombination sites.

The benefits and advantages of the present invention are further described with appropriate reference to the accompanying diagrammatic exhibits.

### BRIEF DESCRIPTION OF THE DRAWINGS

FIGS. 1A-1C are schematic diagrams of a multi-gene assembly vector system consisting of three plasmid vectors (A, B and C), where A is an acceptor assembly process for recombination of a first gene (Gene 1) or an odd ordinal gene;

FIG. 2B is a schematic diagram depicting the second cycle of the gene assembly process for recombination of a second gene (Gene 2) or an even ordinal gene;

FIG. 3 is a schematic diagram depicting the construction of the acceptor vector pYLTAC747;

FIG. 4 is a schematic diagram depicting the construction of the donor vectors pYLVS and pYLSV;

FIG. 5 is an electrophoresis diagram of multi-gene constructs containing different numbers of genes in the acceptor vector pYLTAC747, which were digested by a restriction endonuclease NotI;

FIG. 6 is a schematic diagram of a multi-gene construct pYLTAC747-10G in which 10 genes or DNA segments were stacked; and

FIG. 7 is a photographic diagram of the Southern hybridization detection of multiple genes in transgenic rice plants transformed with the multi-gene vector construct pYLTAC747-10G.

### DETAILED DESCRIPTION OF THE INVENTION

Theoretically, genes carried on separate donor and acceptor vectors can be linked together in an integrative vector indefinitely by multiple rounds of co-integration events using a recombination system. The donor vectors and acceptor vectors each comprises a backbone sequence contained a recombination site, an origin sequence for replication and a bacterial selection marker. However, the backbone sequence of the donor vector must be removed from the integrative vector prior to subsequent round of vector recombination after the first round of recombination. This backbone removal is necessitated because: (i) the doubled replication origins cause instability of the integrative vector in bacteria, and the direct-repeated recombination sites result in reverse recombination; and (ii) a new selection marker gene will be needed in each subsequent round of recombination if the marker gene on the donor vector is not deleted from the integrative vector.

Therefore, two important technical issues must be resolved for multiple cycles of gene recombination. Specifically, (i) appropriately positioned cutting sites in the vectors must be available for the backbone removal in each round of gene recombination; and (ii) the cutting sites for removing the donor backbone must not occur elsewhere within the backbone of the acceptor vector and its growing inserted genes. However, if the cutting sites are not destroyed after each recombination round, the same kinds of sites cannot be used in the subsequent rounds of recombination. When the number of recombined genes increases, the availability of suitable cutting sites decreases.

Endonuclease for genetic engineering with low frequency of recognition sites in genomes is usually called “rare-cutting” endonuclease. Among those, homing endonucleases or meganuclease, such as I-SecI, I-Ceul, I-PpoI, I-TliI, PI-SceI (VDE) and PI-PspI, are very-rare-cutting enzymes. The recognition sequences of homing endonucleases have the following characteristics. (i) The recognition sequences are much longer in bases than those of restriction endonucleases. For example I-SceI and PI-SceI recognize 18-base-pair and 39-base-pair sites, respectively; so the theoretical cutting frequency of natural DNA sequences by the enzymes is very low. (ii) The recognition sequences are asymmetric (Belfort & Roberts, 1997). When two reverse-directed cutting sites are digested by a homing endonuclease and the two ends are subsequently ligated, the joining site no longer contains a complete recognition sequence of the endonuclease.

Some recombination reactions of most recombination systems are reversible. However, some reversible site-specific recombination systems may be modified to produce irreversible recombination (Albert et al., 1997), like that of the attB/attP system mediated by lambda integrase. Irreversible recombination can be used to remove irreversible DNA fragments between two recombination sites, and the two ends can be combined at the same time.

To address the foregoing issues raised above, the present invention provides a method and a multi-gene assembly vector system for effective assembly of multiple genes into a single vector. The vector system consists of an acceptor vector and at least two donor vectors. The method comprises a DNA recombination system allowing multiple rounds of gene recombination by sequential DNA delivery into the acceptor vector via DNA swapping between the acceptor vector and different donor vectors. Multiple donor vectors will be rotatively used in different rounds of recombination to allow sequential insertion of genes or DNA fragments into the acceptor vector.

During the plasmid recombination rounds, specially designed DNA sequences including cutting sites for rare-cutting endonucleases or irreversible recombination sites on the acceptor and donor vectors allow removal of the backbone fragments of the donor vector from the integrative plasmid intermediate in each round of recombination. The method allows continual cycling of gene recombination until all target genes or DNA fragments subcloned in the donor vectors are delivered into the acceptor vector.

In a preferred embodiment of the method, the vectors described in the present invention have two kinds of homing endonucleases cutting sites for the removal of unwanted donor vector backbone fragments, so that cutting of the combined genes or the acceptor vector is avoided. A similar effect can also be achieved using recognition sites for other rare-cutting endonucleases or irreversible recombination sites. By alternate use of the two donor vectors, just two kinds of endonuclease cutting sites or irreversible recombination sites are enough for the multiple rounds of the gene recombination.

The acceptor vector according to the present invention is a recipient of foreign genes or DNA fragments to be delivered, which is characterized by

(1) having a site RS for DNA recombination and can be but is not limited to loxP, FRT, Rs, attB, attP, or Gix;

(2) having a site S1 located near the site RS and is a cutting site for a homing endonuclease or a restriction endonuclease or a site for irreversible recombination;

(3) having a selection marker gene, which can be but is not limited to be an antibiotic resistance gene; and

(4) that a replican for replication is capable of maintaining a large plasmid, which can be but is not limited to bacteriophage P1 replicon, F-factor replicon, Ri replicon, or pVS1 replicon.

The donor vectors in accordance with the present invention are intermediate vectors for transfer of genes of interest to the acceptor vector through gene recombination. The two donor vectors are a donor vector named donor vector I and another donor vector named donor vector II, and are characterized by:

(1) having a site RS for DNA recombination which is the same site RS as in the acceptor vector or can form a recombination with the RS in the acceptor vector;

(2) having a site S1 and another site S2, which are cutting sites for homing endonucleases, rare-cutting restriction endonucleases or irreversible recombination;

(3) that the sites RS, S1, S2 and multi-cloning site (MCS) are located on donor vector I in a relative order of RS-S2-MCS-S1, and on donor vector II in a relative order of RS-S1-MCS-S2.

(4) that the donor vector I and donor vector II each has a selection marker gene, which is different from that in the acceptor vector, and can be but is not limited to be an antibiotic resistance gene and can be the same or different in the donor vectors.

The foregoing homing endonuclease sites can be but are not limited to I-Sce I, I-CeuI, I-PpoI, I-TliI, PI-SceI (VDE) or PI-PspI.

The foregoing irreversible specific recombination sites can be but are not limited to attB, attP, modified attB, modified attP, modified loxP, modified FRT, modified Rs or modified Gix.

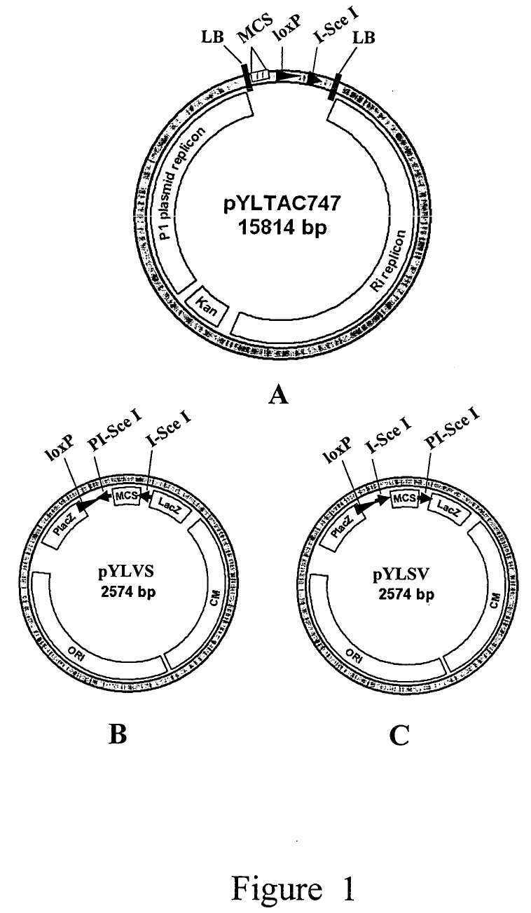

With reference to FIG. 1, an example of a multi-gene assembly vector system in accordance with the present invention includes three vectors (A, B and C). Vector A is an acceptor vector named pYLTAC747, vector B is donor vector I named pYLVS, and vector C is donor vector II named pYSV. In the three vectors (A, B and C), the site RS is represented by loxp, the site S1 by I-SecI, the site S2 by PI-SceI, and MCS is a multi-cloning site consisting of 23 unique restriction endonuclease cutting sites for cloning foreign genes. In the donor vectors (B, C), LacZ is a galactosidase gene as a selection marker for cloning, and Cm is a chloramphenicol-resistance selection marker gene. In the acceptor vector (A), Kan is a kanamycin-resistance selection marker gene. RB and LB are respectively the right and left borders of the transfer DNA region (T-DNA). The P1 plasmid replicon in the acceptor vector (A) is originally taken from the bacteriophage P1, which makes the plasmid replicate in E. coli. The Ri replicon in the acceptor vector (A) is originally taken from Agrobacterium rhizogenes Ri plasmid and makes the plasmid replicate in Agrobacterium rhizogenes and Agrobacterium tumefaciens. Ori in the donor vectors (B, C) is a pUC plasmid replication origin.

The method to assembly multiple genes in accordance with the present invention comprises the steps of (1) cloning target genes or DNA fragments into separate donor vectors, (2) performing plasmid recombination of donor vector I and the acceptor vector, (3) performing plasmid recombination of donor vector II and the acceptor vector and (4) repeating the second and third steps until all desired target genes and DNA fragments are transferred to the acceptor vector.

Target genes or DNA fragments are cloned by conventional cloning techniques into the MCS of separate donor vectors to make the genes inserted between the sites S1 and S2, in the order the genes are to be combined in the acceptor vector. If technically possible, two or more genes may be cloned as a gene group into a donor vector. The word “gene” as used in the detailed description represents either a functional gene or a DNA fragment.

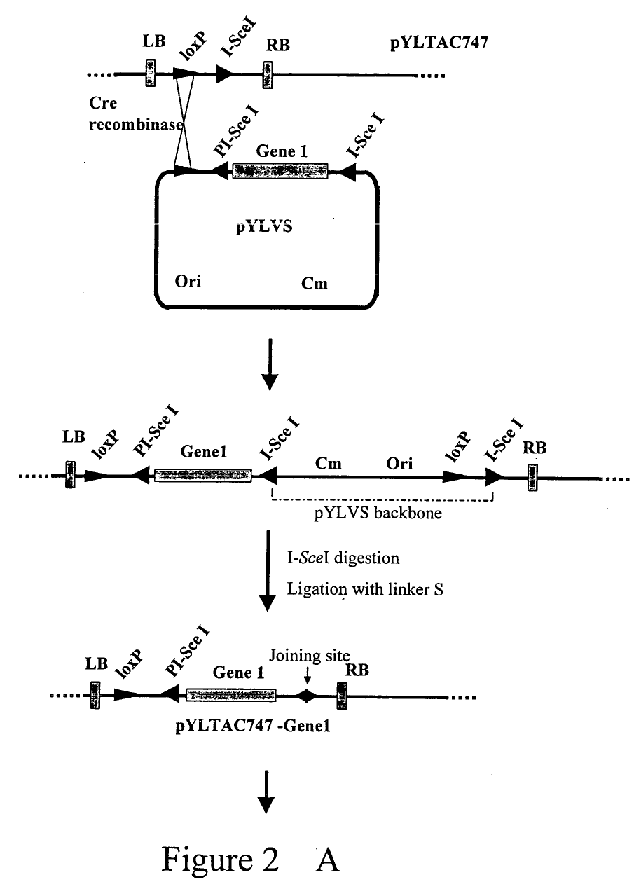

With reference to FIG. 2A, the donor vector I plasmid containing the first gene or gene group (pYLVS-Gene 1) and acceptor vector plasmid pYLTAC747 are co-transferred into an Escherichia coli (E. coli) host that expresses the Cre recombinase which catalyzes the plasmid recombination in vivo. The transformants are then selected on selection medium containing kanamycin and chloramphenicol. The plasmids consisting of integrated and separate plasmids are purified, and then re-transferred into an E. coli host that does not have the Cre gene, and the transformants are selected on selection medium containing kanamycin and chloramphenicol. The plasmid recombination also can be carried out in vitro with purified Cre recombinase. A backbone fragment of pYLVS flanked by the two I-SecI sites is removed by digestion with endonuclease I-SceI. Since the two asymmetric I-SceI sites on the co-integrated plasmid are arranged in opposite orientation, the protruding 3′ ends are not complementary to each other, and the plasmid circularization by T4 DNA ligase reaction formed a joining site requires the aid of a compatible double-stranded oligo-nucleotide linker S. The resulting joining site on the plasmid is no longer recognized by the enzyme during subsequent gene-assembly cycles. The resulted plasmid bearing Gene 1 is transferred into E. coli. Transformants are selected on selection medium containing kanamycin, and then tested for chloramphenicol-sensitivity. The selected clone is a new plasmid without the pYLVS backbone fragment but with Gene 1 inserted, which is named pYLTAC747-Gene 1. On pYLTAC747-Gene 1, the homing endonuclease cutting site is replaced by a PI-SceI site that is derived from pYLVS.

With reference to FIG. 2B, the donor vector II plasmid containing the second gene or gene group (pYLSV-Gene 2) and the acceptor vector plasmid pYLTAC747-Gene 1 are co-transferred into an E. coli host that expresses the Cre recombinase. Transformants are selected on selection medium containing kanamycin and chloramphenicol. The plasmids are purified, re-transferred into an E. coli host that does not have the Cre gene and selected on selection medium containing kanamycin and chloramphenicol. The plasmid recombination also can be carried out in vitro with purified Cre recombinase. A backbone fragment of pYLSV flanked by the two PI-SceI sites is removed by digestion with endonuclease PI-SceI. Since the two asymmetric PI-SecI sites on the co-integrated plasmid are arranged in opposite orientation, the protruding 3′ ends are not complementary to each other, and the plasmid circularization by ligation formed a joining site requires the aid of a compatible double-stranded linker V. After ligation with T4 DNA ligase, the resulting joining site on the plasmid is no longer recognized by the enzyme during subsequent gene-assembly cycles. The resulted plasmid bearing Gene 1 and Gene 2 is transferred into E. coli. Transformants are selected on selection medium containing kanamycin and then tested with chloramphenicol for chloramphenicol-sensitivity. The selected clone is a new plasmid without the pYLSV backbone fragment but with Gene 2, which is named pYLTAC747-Gene1-Gene2. The cutting site for homing endonuclease on this new plasmid becomes I-SceI, the same as that on the original vector pYLTAC747.

The second and third steps described above are repeated alternately with the donor vectors I and II containing new target genes for the gene recombination until all target genes are delivered into the acceptor vector.

The I-SceI recognition sequence and cutting point (arrowed) are as follows:

5′-TAGGGATAA↓CAGGGTAAT-3′
3′-ATCC↑CTATTGTCCCATTA-5′

Two reverse-directed I-SceI cutting ends and an oligo-nucleotide linker S (presented by lowercase letters) are combined to produce a joining site as follows:

5′-TAGGGATAAnnn . . . nnnttatCCCTA-3′
3′-ATCCCtattnnn . . . nnnAATAGGGAT-5′

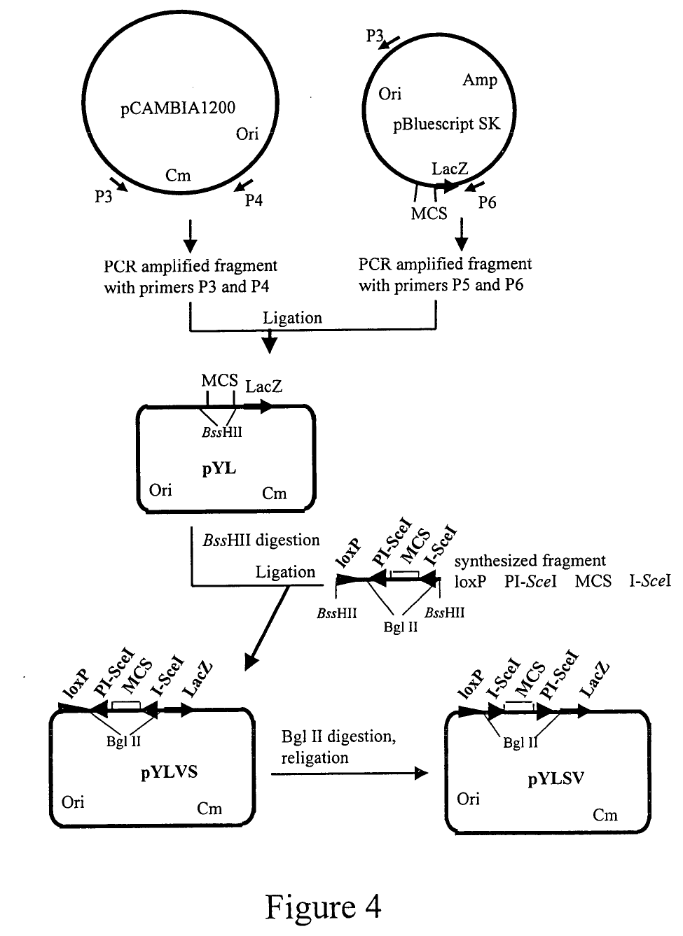

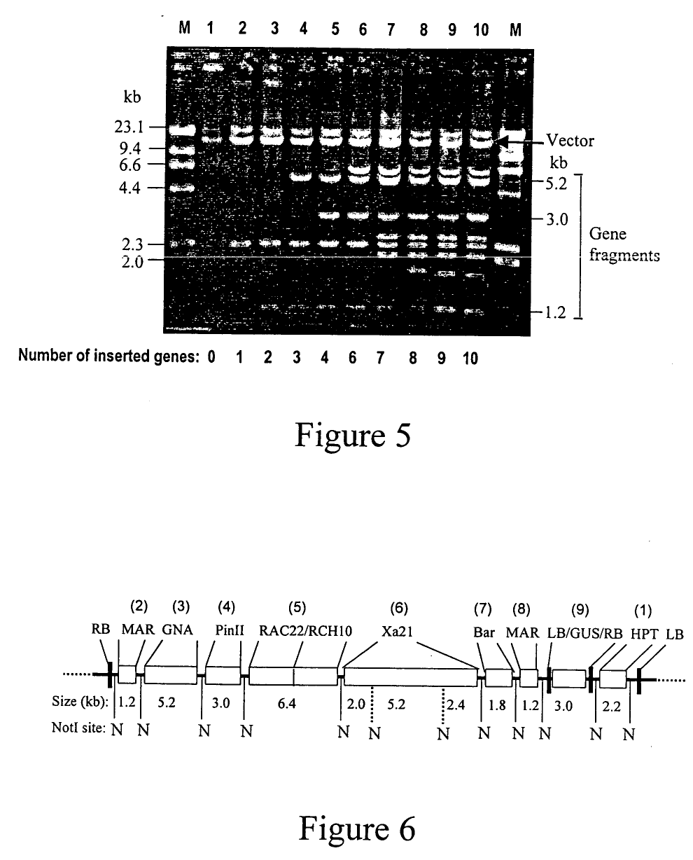

The base number of the linker core sequence (n) is preferably eight or more, and the linker core sequence (n) can be any bases but cannot form a complete I-SecI or PI-SecI recognition site. In an example (FIG. 5 and FIG. 6) of the present invention, a restriction site NotI is designed in the linker S:

5′-gcggccgcttat-3′
3′-tattcgccggcg-5′

The PI-SecI recognition sequence and cutting point (arrowed) are as follows:

5′-ATCTATGTCGGGTGC↓GGAGAAAGAGGTAATGAAATGGCA-3′
3′-TAGATACAGCC↑CACGCCTCTTTCTCCATTACTTTACCGT-5′

Two reverse-directed PI-SecI cutting ends and an oligo-nucleotide linker V (presented by lowercase letters) are combined to produce a joining site as follows:

5′-ATCTATGTCGGGTGCnnn . . . nnngcacCCGACATAGAT-3′
3′-TAGATACAGCCcacgnnn . . . nnnCGTGGGCTGTATCTA-5′

The base number of the linker core sequence (n) is preferably eight or more, and the linker core sequence (n) can be any bases but cannot form a complete PI-SecI or I-SceI recognition site. In an example (FIG. 5 and FIG. 6) of the present invention, a restriction site NotI is designed in the linker V:

5′-gcggccgcgcac-3′
3′-cagccgccggcg-5′

Accordingly, a method for producing a recombinant vector construct in accordance with the present invention comprises providing an acceptor vector and a donor vector, introducing the acceptor vector and the donor vector into cells allowing occurrence of DNA recombination, subjecting the cells to drug selection, obtaining a recombinant vector, subjecting the recombinant vector to endonuclease digestion and drug selection and obtaining a recombinant acceptor vector.

The acceptor vector provided comprises a sequence for DNA recombination or called DNA recombination sequence, a selection marker gene and a first endonuclease cutting site. The first endonuclease cutting site is flanked by the sequence for DNA recombination and is unique in the acceptor vector. The selection marker gene is flanked by the first endonuclease cutting site.

The donor vector provided comprises a DNA recombination sequence, a target sequence of interest to be delivered into the acceptor vector, a selection marker gene, a first endonuclease cutting site and a second endonuclease cutting site. The first endonuclease cutting site and the second endonuclease cutting site are each unique in the donor vector. The DNA recombination sequence can form a recombination with the corresponding sequence in the acceptor vector. The DNA recombination sequence is flanked by the second endonuclease cutting site. The target sequence of interest is flanked by the second endonuclease cutting site and the first endonuclease cutting site. The selection marker gene is flanked by the first endonuclease cutting site and is different from the selection marker gene in the acceptor vector.

The acceptor vector and the donor vector are introduced into cells to allow plasmid recombination between the acceptor vector and the donor vector carrying the target sequence. The cells are subjected to drug selection with respect to the two different selection marker genes in the acceptor vector and the donor vector, respectively. A recombinant vector is obtained from the cells surviving from the drug selection. The recombinant vector is subjected to endonuclease digestion with the first endonuclease followed by self-ligation to form a circular recombinant plasmid. The recombinant plasmid is subjected to drug selection with the selection marker gene in the acceptor vector. A recombinant acceptor vector surviving from the drug selection is obtained. The recombinant acceptor vector comprises the target sequence of interest from the donor vector, the second endonuclease cutting site, the selection marker gene in the acceptor vector and the DNA recombination sequence.

Preferably, the method for producing a multi-gene recombinant vector construct further comprises repeating one or more times the steps of providing an additional donor vector carrying a target sequence of interest, introducing the recombinant acceptor vector and the additional donor vector into cells to allow DNA recombination, subjecting the cells to drug selection, obtain a recombinant vector, subjecting the recombinant vector to endonuclease digestion and self-ligation, and drug selection and obtaining a new recombinant acceptor vector.

The additional donor vector provided comprises a DNA recombination sequence, a target sequence of interest to be delivered into the acceptor vector, a selection marker gene, a first endonuclease cutting site and a second endonuclease cutting site. The first endonuclease cutting site and the second endonuclease cutting site are each unique in the donor vector. The DNA recombination sequence can form a recombination with the corresponding sequence in the acceptor vector. The DNA recombination sequence is flanked by the first endonuclease cutting site. The target sequence of interest is flanked by the first endonuclease cutting site and the second endonuclease cutting site. The selection marker gene is flanked by the second endonuclease cutting site and is different from the selection marker gene in the acceptor vector.

The recombinant acceptor vector and the additional donor vector are introduced into cells to allow occurrence of plasmid recombination between the acceptor vector and the donor vector carrying the target sequence. The cells are subjected to drug selection with respect to the two different selection marker genes in the recombinant acceptor and the additional donor vector, respectively. A recombinant vector is obtained from the cells surviving from the drug selection. The recombinant vector is subjected to endonuclease digestion with the a endonuclease that cut the same endonuclease cutting site in the recombinant acceptor vector, followed by self-ligation to form a circular recombinant plasmid. A recombinant acceptor vector surviving the drug selection is obtained. The recombinant acceptor vector comprises the target sequence of interest in the additional donor vector.

The acceptor vector may comprise all or part of DNA sequence SEQ ID NO: 1 (see below). The first donor vector may comprise all or part of DNA sequence SEQ ID NO: 2 (see below). The second donor vector may comprise all or part of DNA sequence SEQ ID NO: 3 (see below).

Other possible modifications and variations can be made without departing from the spirit and scope of the present invention as claimed in the invention. Such modifications may concern but are not limited to the number of donor vectors or the number and/or arrangements of the specific sites for recombination and digestion. For example, three or more donor vectors can be used in turn to recombine with the acceptor vector. In the case of using three donor vectors, the sites and their location orders on the acceptor vector and the donor vectors can be designed as follows:

acceptor vector: RS-S1

donor vector I: RS-S2-MCS-S1

donor vector II: RS-S3-MCS-S2

donor vector III: RS-S1-MCS-S3

Herein RS is a recombination site. S1, S2 and S3 are cutting sites for homing endonucleases or rare-cutting endonucleases or irreversible recombination sites. During multi-gene assembly cycling, each donor vector is used in turn in the order of donor vector I, donor vector II, donor vector III, donor vector I, donor vector II and so on.

The multi-gene assembly method in accordance with the present invention has several applications. The present invention can preferably be used to construct multi-gene transformation vectors suitable for various transformation methods, so that multiple genes can be transferred together into various recipient organisms including but not limited to plants, animals, insects, yeast and micro-organisms, for the purposes of production of multiple gene-products or expression of characters based on interactions of multiple genes. Transformation methods for transfer of multiple genes with constructs made according to the present invention comprises but are not limited to the Agrobacterium-mediated transformation method, particle bombardment, micro-injection, electroporation, Polyethylene Glycol method, pollen-tube pathway transformation method or viral mediated gene transformation method. For example, the acceptor vector pYLTAC747 described in the present invention as an example contains all components of a binary transformation vector needed for Agrobacterium-mediated transformation, i.e., the right and left borders of a T-DNA region, the bacteria antibiotic selection marker (kanamycin-resistance gene) and the P1 and Ri plasmid replicons functional in E. coli and Agrobacterium. After insertion of a plant selection marker gene and other target genes into pYLTAC747 with the method of the present invention, the vector constructs can be used for transformation of plants by Agrobacterium-mediated transformation or other transformation methods. Using the method in accordance with the present invention, various types of vectors can be modified easily as the acceptor and donor vectors suitable for assembly of multiple genes or DNA fragments to construct various types of genetic engineering vectors for specific purposes, especially large-size or intricate vectors containing multiple elements, for example bacterial artificial chromosomes, yeast artificial chromosomes, mammalian artificial chromosomes or plant artificial chromosomes.

The present invention has the following advantages:

The method is flexible and versatile. Multiple genes or DNA fragments of different sources can be effectively combined into one vector, and the placement and orientation of target genes in the vector can be freely designed and readily achieved in a reliable step-by-step process. The present invention overcomes the technical limitations of existing cloning methods for producing multi-gene vector constructs.

By alternately using two donor vectors with an acceptor vector for gene recombination, multiple cycles of gene recombination can be repeated. With this strategy, just two rare-cutting sites for endonuclease or irreversible specific recombination sites are enough to remove the unneeded backbone fragments of the donor vectors, which is a necessary step for multiple rounds of gene recombination.

Using replicons with the ability to maintain a large plasmid, such as the P1 plasmid replicon and the Ri replicon, the acceptor vector described in the present invention can accept and stably maintain a large number of foreign genes.

All of the documents or publications recited in the text are incorporated herein for reference.

### EXAMPLES

The present invention is further described in specific detail by reference to the following examples showing construction of the multi-gene assembly vector system and its application to introduce multiple genes into rice. However, the claims of the present invention are not limited by these examples.

### Example 1

This example shows the construction of the acceptor vector pYLTAC747.

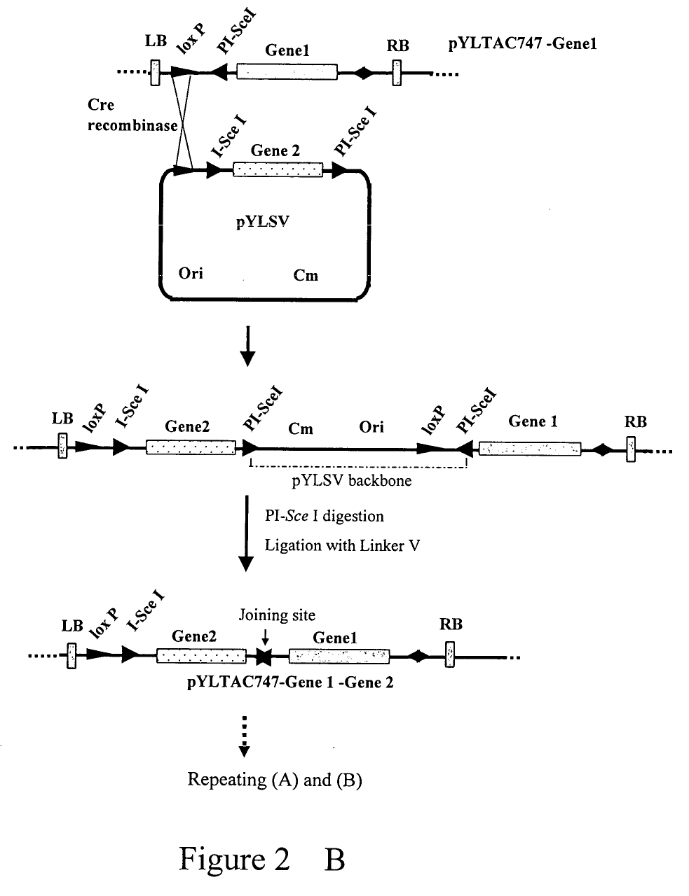

With reference to FIG. 3, Primer P1 is:

5′-CTCATGTCTAGATTGTCGTTTCCCGCCTTCAGT-3′, the underlined sequence is a XhaI restriction site.

Primer P2 is:

5′-ACCGGATCCTGTTTACACCACAATATATCCTGCCACGTTAAAGACTTCAT-3′, the underlined sequence is a BamHI restriction site, and the italicized sequence is the left border LB of T-DNA.

The fragment MCS-loxP-I-SceI fragment (SEQ ID NO: 1) is: 5′-GGATCCAAGCTTGTCGACGGCCGGCCGCGGCCGCATAACTTCGTATAG CATACATTATACGAAGTTATGGGCCGCattaccctgttatccctaGGCCCCAATTAG GCCTACCCACTAG-3′. The underlined sequence is the multiple cloning site (MCS) composed of BamHI, HindIII, FseI and NotI. The italicized sequence is the LoxP site. The lowercase letters are an I-SecI site.

The primers P1 and P2 containing XbaI and BamHI sites were synthesized according the transformation-competent artificial chromosome vector pYLIAC7 sequence (Liu et al., 1999). A vector frame fragment (15690 bp) was amplified by PCR and digested with XbaI and BamHI and ligated with the synthesized double-stranded DNA fragment MCS-loxP-I-SceI (SEQ ID NO: 1) to produce an acceptor vector plasmid, which was named pYLTAC747.

### Example 2

This example shows the construction of the donor vectors.

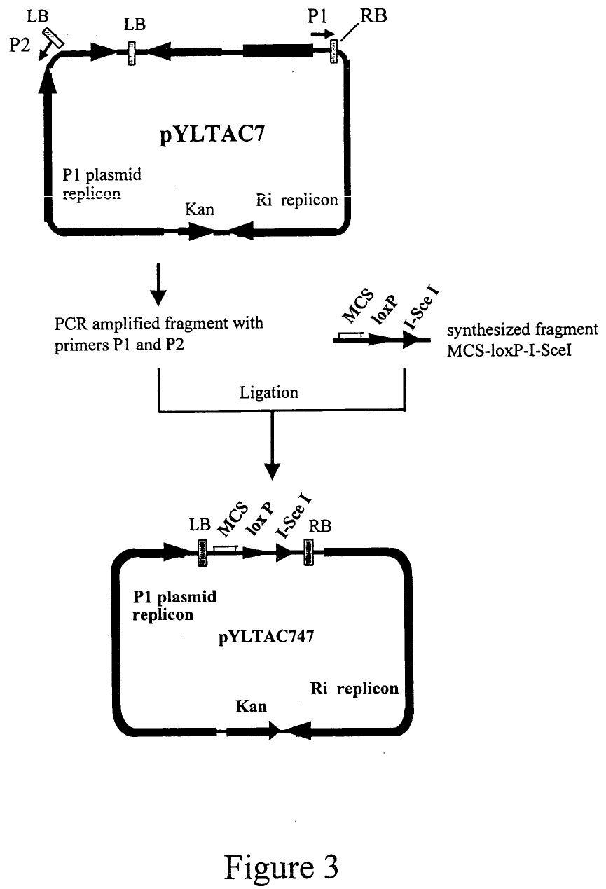

With reference to FIG. 4, pCAMBIA1200 and pBluescript SK were plasmid vectors. Ori is a plasmid replication origin. Cm is a chloramphenicol-resistance gene. Amp is an ampicillin-resistance gene. LacZ is a galactosidase gene as a selection marker.

Primer 3 is 5′-CTTCAATATTACGCAGCA-3′
Primer 4 is 5′-GAGCAATATTGTGCTTAG-3′
Primer 5 is 5′-GTTCTCGCGGTATCATTG-3′
Primer 6 is 5′-CCATTCGCCATTCAGGCTG-3′

The sequence loxP-PI-SceI-MCS-I-SceI region in the donor vector I plasmid pYLVS (SEQ ID NO: 2) is: 5′-

GCGCGCTCATAACTTCGTATAGCATAC
ATTATACGAAGTTATCAGATCTTTTTGGCTACCTTAAGTGCCATT
TCATTACCTCTTTCTCCGCACCCGACATAGATGTTAAGAGAGTCATAT
CGATGCATGCGGCCGCTAGCTCGAGCTCTAGAATTCTGCAGGTACCG
CGGATCCATGGGCCCGGGACTAGTCGACATGTACAAGCTTGtagggataa
acagggtaatCCCTAAGATCTCAGCGCGC-3′

The sequence loxP-I-SceI-MCS-PI-SceI in the donor vector II plasmid pYLSV (SEQ ID NO: 3) is: 5′-

GCGCGCTCATAACTTCGTATAGCATACATTATACGAAGTTATCAGATCTT
AGGGattaccctgttatccctaCAAGCTTGTACATGTCGACTAGTCCCGG
GCCCATGGATCCGCGGTACCTGCAGAATTCTAGAGCTCGAGCTAGCGGCC
GCATGCATCGATATGACTCTCTTAACATCTATGTCGGGTGCGGAGAAAGA
GGTAATGAAATGGCACTTAAGGTAGCCAAAAAGATCTCAGCGCGC-3′

The italicized sequence is a loxP site. The underlined sequence is a PI-SceI site. The lowercase sequence is an I-SecI site. The sequence between the PI-SceI site and the I-SceI site is a multi-cloning site (MCS) consisting of 23 unique restriction sites.

The primers P3 and P4 were synthesized based on the sequence of the chloramphenicol-resistance gene. A chloramphenicol-resistance gene Cm (826 bp) was amplified by PCR from plasmid pCAMBIA1200. The primers P5 and P6 were synthesized based the plasmid pBluescript SK sequence, and a fragment (Ori-MCS-LacZ) of 1660 bp was amplified by PCR from the plasmid pBluescript SK. The two fragments were ligated and transferred to E. coli DH10B to obtain an intermediate plasmid pYL. The original MCS in plasmid pYL that was derived from pBluescript SK was removed by digestion with restriction endonuclease BssHII, and the synthesized double-stranded DNA fragment loxP-PI-SceI-MCS-I-SceI (SEQ ID NO: 2) was inserted into the plasmid by ligation to form a new plasmid. This plasmid was donor vector I and was named pYLVS. Two restriction sites BglII were designed on the vector, one located between loxP and PI-SceI, and the other located between I-SecI and LacZ. Therefore another donor vector (donor vector II) was produced from pYLSV by digestion with BglII and re-ligation to insert the BglII-fragment containing the I-SceI-MCS-PI-SceI sites back into the vector in the opposite orientation. In donor vector II, the relative locations of the sites were changed to LoxP-I-SceI-MCS-PI-SceI (SEQ ID NO: 3), and this vector was named pYLSV.

### Example 3

This example illustrates the construction of vector constructs containing multiple genes for plant transformation.

With reference to FIG. 5, multiple vector constructs containing different number genes were digested with a restriction endonuclease NotI and subjected to gel electrophoresis. The figures at the bottom of FIG. 5 indicated the number of target genes and functional DNA fragment delivered into the vector pYLTAC747 during the following multi-gene assembly process. The 5.2 kb band in lanes 7-10, 1.2 kb band in lanes 9-10 and 3.0 kb band of lane 10 are bands with two co-migrating fragments (see FIG. 6 for the sizes of the Not I fragments). Lane M is a lambda DNA/HindIII molecular weight marker.

Genes and functional DNA sequences used for the recombination were hygromycin-resistance gene (HPT), matrix attachment region (MAR), snow drop lectin gene (Galanthus nivalis agglutinin) (GNA), potato proteinase inhibitor II (PinII), rice acidic chitinase (RAC22), rice basic chitinase (RCH10), rice bacterial blight resistance gene (Xa21), and beta-glucuronidase gene (GUS). These genes were originally individually cloned in plasmid vector pBluescript SK+or pUC18.

The first gene HPT was directly cloned into the NotI site of pYLTAC747 by conventional cloning methods. The produced vector pYLTAC747-HPT was presented in FIG. 5 lane 2.

The MAR sequence (1.2 kb) was sub-cloned into the donor vector I plasmid pYLVS to produce pYLVS-MAR. The pYLVS-MAR and pYLTAC747-HPT were used to co-transform E. coli NS3529 that contains the Cre gene and expressed Cre recombinase, and formed a recombined plasmid. The recombined plasmid was selected on an LB medium containing kanamycin and chloramphenicol, purified and re-transferred to E. coli DH10 B lacking the Cre gene. The integrated plasmid was subjected to I-SceI digestion to cut off the pYLVS backbone. The digested plasmid was ligated with an oligo-nucleotide linker S (containing a NotI site) with T4 DNA ligase to form a circular plasmid. After testing for chloramphenicol-sensitivity, a new plasmid named pYLTAC747HPT-MAR was obtained (FIG. 5 lane 3).

The GNA gene (5.2 kb) was subcloned into the donor vector II pYLSV to produce pYLSV-GNA. The pYLSV-GNA and pYLTAC747-HPT-MAR were used to co-transform NS3529 to form a recombined plasmid. The recombined plasmid was selected on LB medium containing kanamycin and chloramphenicol, purified and re-transferred to DH10B. The integrated plasmid was purified and subjected to PI-SceI digestion to cut off the pYLSV backbone. The digested plasmid was ligated with an oligo-nucleotide linker V (containing a NotI site) with T4 DNA ligase to form a circular plasmid. After testing for chloramphenicol-sensitivity, a new plasmid named pYLTAC747HPT-MAR-GNA was obtained (FIG. 5 lane 4).

The PinII gene (3.0 kb) was subcloned into pYLVS to produce pYLVS-PinII. The pYLVS-PinII and pYLTAC747-HPT-MAR-GNA were used to co-transform to NS3529 to form a recombined plasmid. The recombined plasmid was selected on LB medium containing kanamycin and chloramphenicol, purified and re-transferred to DH10B. The integrated plasmid was subjected to I-SceI digestion to cut off the pYLVS backbone, and ligated with the linker S with T4 DNA ligase to form a circular plasmid. After testing for chloramphenicol-sensitivity,a new plasmid named pYLTAC747-HPT-MAR-MAR-GNA-PinII was obtained (FIG. 5 lane 5).

The rice genes, RAC22/RCH10 genes (6.4 kb) that were originally cloned to the same plasmid vector, were subcloned into pYLSV to produce pYLSV-RAC22/RCH10. The pYLSV-RAC22/RCH10 and pYLTAC747-HPT-MAR-GNA-PinII were used to co-transform NS3529, and formed a recombined plasmid. The recombined plasmid was selected on an LB medium containing kanamycin and chloramphenicol, purified and re-transferred to DH10B. The integrated plasmid was purified and subjected to PI-SecI digestion to cut off the pYLSV backbone. The digested plasmid was ligated with the oligo-nucleotide linker V with T4 DNA ligase to form a circular plasmid. After testing for chloramphenicol-sensitivity, a new plasmid named pYLTAC747-HPT-MAR-GNA-PinII-RAC22/RCH10 was obtained (FIG. 5 lane 6).

The Xa21 gene (9.7 kb) was subcloned into pYLVS to produce pYLVS-Xa21. The pYLVS-Xa21 and pYLTAC747-HPT-MAR-GNA-PinII-RAC22/RCH10 were used to co-transform NS3529, and form a recombined plasmid. The recombined plasmid was selected on LB medium containing kanamycin and chloramphenicol, purified and re-transferred to DH10B. The integrated plasmid was purified and subjected to I-SceI digestion to cut off the pYLVS backbone. The digested plasmid was ligated with the oligo-nucleotide linker with T4 DNA ligase to form a circular plasmid. After testing for chloramphenicol-sensitivity, a new plasmid named pYLTAC747-HPT-MAR-GNA-PinII-RAC22/RCH10-Xa21 was obtained (FIG. 5 lane 7, note that the Xa21 gene does not have two NotI sites inside).

The Bar gene (1.8 kb) was sub-cloned into pYLSV to produce pYLSV-Bar. The pYLSV-Bar and pYLTAC747HPT-MAR-GNA-PinII-RAC22/RCH10-Xa21 were used to co-transform NS3529, and formed a recombined plasmid. The recombined plasmid was selected on LB medium containing kanamycin and chloramphenicol, purified and re-transferred to DH10B. The integrated plasmid was purified and subjected to PI-SceI digestion to cut off the pYLSV backbone. The digested plasmid was ligated with the oligo-nucleotide linker V with T4 DNA ligase to form a circular plasmid. After testing for chloramphenicol-sensitivity, a new plasmid pYLTAC747-HPT-MAR-GNA-PinII-RAC22/RCH10-Bar was obtained (FIG. 5 lane 8).

The plasmid pYLVS-MAR previously obtained and pYLTAC747HPT-MAR-GNA-PinII-RAC22/RCH10-Xa21-Bar were used to co-transform E. coli NS3529 to form a recombined plasmid. The plasmid was selected on LB medium containing kanamycin and chloramphenicol, purified and re-transferred to DH10B. The plasmid was purified and subjected to I-SecI digestion to cut off the pYLVS backbone. The digested plasmid was ligated with the oligonucleotide linker S with T4 DNA ligase to form circular plasmid. After testing for chloramphenicol-sensitivity, a new plasmid pYLTAC747-HPT-MAR-GNA-PinII-RAC22/RCH10-Bar-MAR was obtained (FIG. 5 lane 9).

The GUS gene flanked by LB and RB (LB/GUS/RB, 3.0 kb) was subcloned into pYLSV to produce pYLSV-LB/GU/RB. The pYLSV-LB/GUS/RB and pYLTAC747-HPT-MAR-GNA-PinII-RAC22/RCH10-Xa2 1 -Bar-MAR were used to co-transform NS3529, and formed a recombined plasmid. The recombined plasmid was selected on LB medium containing kanamycin and chloramphenicol, purified and re-transferred to DH10B. The integrated plasmid was purified and subjected to PI-SceI digestion to cut off the pYLVS backbone, and ligated with the linker V with T4 DNA ligase to form a circular plasmid. After testing for chloramphenicol-sensitivity, a new plasmid named pYLTAC747-HPT-MAR-GNA-PinII-PAC22/PCH10-Xa21-Bar-MAR-LB/GUS/RB was obtained (FIG. 5 lane 10).

A final construct contained ten foreign genes and functional DNA sequences and was re-named pYLTAC747-10G. The structure of the gene between RB and LB was shown in FIG. 6. The figures in blanket were the order of the genes or DNA sequences being inserted to the vector. N denotes NotI sites derived from the linker S and linker V or existed in the vector and the Xa21 gene. The figures present between NotI sites indicate the fragment length (kb). This example proves that the method disclosed in the present invention is effective for assembly of multiple genes and DNA sequences of different sources in a vector.

### Example 4

This example shows the effectiveness of the multi-gene transformation vector for transfer of multiple genes together into rice genome.

The plasmid pYLTAC747-10G was transferred to A. tumefaciens EHA105 to obtain Agrobacterium clone EHA 105[pYLTAC747-10G]. EHA105[pYLTAC747-10G] was used to transform rice callus tissue. pYLTAC747-10G contains the HPT and Bar genes that can be used for selection of transformants with hygromycin and/or herbicide Basta. Rice embryos were inoculated to a medium to induce calli at 25° C. in dark. The induced calli was transferred to subculture medium. The EHA 105[pYLTAC747-10G] cells were cultured on YM agar medium at 28° C. for 24 hours, followed by culture in 40 ml MB liquid medium containing 100 μmol/L acetosyingone at 28° C. until OD₅₅₀=0.5-1.0. The calli was inoculated with EHA105 [pYLTAC747-10G] for 20 minutes, transferred to MB agar medium, and cultured at 25° C. for 72 hours in dark. The calli were transferred to a medium containing 50 mg/L hygromycin for selective culture for 4 weeks. After selection, the calli were transferred to a regeneration medium to regenerate plantlets. More than 50 transformed rice plants were obtained.

### Example 5

This example was the detection of transgenes from transformed rice plants by molecular hybridization.

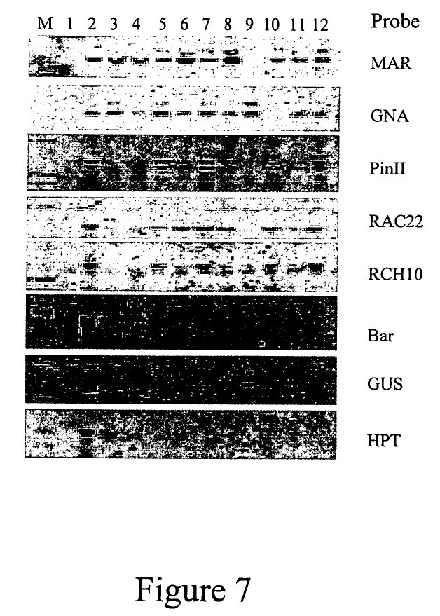

Genomic DNAs were isolated from the T₀ transgenic rice plants and digested by restriction endonuclease HindIII and run onto an agarose gel. After blotting to a hybridization membrane, the transgenes integrated to rice genome were detected using the transgene fragments as probes. As shown in FIG. 7, in most transgenic plants all transgenes present in the same T-DNA region were transferred together into the rice genome. Lane M is a lambda DNA/Hind III molecular weight marker. These results demonstrate that the multi-gene vector constructed according to the present invention can effectively transfer multiple genes into plant genomes.

Although the invention has been explained in relation to its preferred embodiments, many other possible modifications and variations can be made without departing from the spirit and scope of the invention as hereinafter claimed.

References

1. Albert H., Dale E. C., Lee E. and OW D. W. (1995) Plant J., 7:649-659.

2. Belfort M. & Roberts R. J. (1997) Nucl. Acids Res., 25:3379-3388.

3. Chen L., Marmey P., Taylor N. J., et al. (1998) Nat. Biotechnol. 16: 1060-1064.

4. Daniell H., Dhingra A. (2001) Current Opinion in Biotechnology., 13:136-141

5. Halmilton C. M. (1997) Gene, 200:107-116.

6. Halpin C., Barakate A., Askaii B. M., et al. (2001). Plant Mol. Biol., 47:295-310.

7. Lapierre C., Pollet B., Petit-Conil M., et al. (1999) Plant Physiol., 119:153-163.

8. Liu Y. G., Shirano Y., Fukaki H., et al. (1999) Proc. Natl. Acad. Sci. USA., 96:6535-6540.

9. Ma J. K. Hiatt A., Hein M., et al. (1995) Science., 268:716-719.

10. McCormac A. C., Elliott M. C. & Chen D. F. (1999) Mol. Gen. Genet., 261:226-235.

11. Mcleld M., Craft S., & Broach J. R. (1986) Mol. Cell. Biol., 6:3357-3367.

12. Merker P., Muskhelishvili G. (1993) Cold Spring Harbour Symp. Quart, Biol., 58:505-513.

13. Mostov K. & Lehner T. (1995) Science. 268:716-719.

14. Nash H. A. (1981) Ann. Rev. Genet. 15:143-167.

15. Shizuya H., Birren B., et al. (1992) Proc. Natl. Acad. Sci. USA., 89:8794-8797.

16. Sternberg N. & Hamilton D. (1981) J. Mol. Biol., 150:467-486.

17. Sternberg N. (1990) Proc.Natl. Acad. Sci. USA. 87:103-107.

18. van Engelen F A, Schouten A, et al. (1994) Plant Mol Biol. 26:1701 -10.

19. Ye X., Al-Babili S., Kl\ddot{o}ti A., Zhang J., et al. (2000) Science, 287:303-305.

What is claimed is:

1. A method for producing multi-gene recombinant vector constructs, which comprises:
(1) a multi-gene assembly vector system comprising an acceptor vector and at least two donor vectors; and
(2) a DNA recombination system allowing two or more rounds of gene assembly by sequential DNA delivery into the acceptor vector via DNA swapping between the acceptor vector and different donor vectors; and multiple donor vectors will be rotatively used in different rounds of recombination to allow sequential insertion of genes or DNA fragments into the acceptor vector.

2. The method of claim 1, wherein said acceptor vector comprises:
(1) a site RS for DNA recombination;
(2) a site S1 located near said RS, which is a cutting site for a homing endonuclease or a restriction endonuclease, or a site for irreversible specific recombination;
(3) a selection marker gene that is different from that contained in said donor vector; and
(4) a replicon for replication, including those capable of maintaining large plasmids.

3. The method of claim 1, wherein a donor vector I comprises:
(1) a site RS for DNA recombination, which is the same site RS as on said acceptor vector or can form a specific recombination with the RS on said acceptor vector;
(2) a site S1 and another site S2, which are cutting sites for homing endonucleases, or for rare-cutting restriction endonucleases, or sites for irreversible recombination;
(3) a multi-cloning site MCS;
(4) locations of the sites of RS, S1, S2 and MCS on said donor vector in relative order of RS-S2-MCS-S1; and
(5) a selection marker gene different from that contained in said acceptor.

4. The method of claim 1, wherein another donor vector II comprises:
(1) a site RS for DNA recombination, which is the same site RS as on said acceptor vector or can form a specific recombination with the RS on said acceptor vector;
(2) a site S1 and another site S2, which are cutting sites for homing endonucleases, or for rare-cutting restriction endonucleases, or sites for irreversible recombination;
(3) a multi-cloning site MCS;
(4) locations of the sites of RS, S1, S2 and MCS on said donor vector in relative order of RS-S1-MCS-S2; and
(5) a selection marker gene different from that contained in said acceptor vector.

5. The method according to claim 1, wherein said multi-gene assembly vector system comprising an acceptor vector and donor vector I and donor vector II is used to carry out two or more cycles of DNA recombination by alternate use of said donor vector I and donor vector II together with said acceptor vector to construct multi-gene vector constructs, which recombination process comprises the steps of:
(1) cloning of target single genes or gene groups of interest including any DNA fragments by conventional molecular cloning techniques into the MCS of said donor vector I or donor vector II, to make the target gene or genes inserted between the sites S1 and S2;
(2) carrying out the first cycle of DNA recombination to recombine the first target gene or gene groups into said acceptor vector through: (i) in vivo or in vitro plasmid recombination with said donor vector I containing target gene and said acceptor vector, upon double-selection of transformants with the selection marker genes of the plasmids; (ii) removing the backbone sequence of said donor vector I between the two S1 sites from the integrative plasmid by digestion with an endonuclease that cut S1 sites, followed by plasmid circularization by ligation with T4 DNA ligase, and if necessary, with the aid of a double-stranded oligonucleotide linker compatible to the S1 cutting ends; and (iii) if S1 is an irreversible recombination site, performing an in vivo or in vitro recombination reaction with corresponding recombinase, upon which the backbone sequence of said donor vector I between the two S1 sites is removed and the ends of the acceptor vector bearing the inserted gene or gene group are joined to form a circular plasmid;
(3) carrying out the second cycle of DNA recombination to recombine the second target gene or gene groups into said acceptor vector through: (i) in vivo or in vitro plasmid recombination with said donor vector II containing the second gene or gene group and the acceptor vector plasmid obtained from the step (2), upon double-selection of transformants with the selection marker genes of the plasmids; (ii) removing the backbone sequence of the donor vector II between the two S2 sites from the integrative plasmid by digestion with an endonuclease that cut S2 sites followed by plasmid circularization by ligation with T4 DNA ligase, and if necessary, with the aid of a double-stranded oligonucleotide linker compatible to the S2 cutting ends; and (iii) if S2 is an irreversible recombination site, performing an in vivo or in vitro recombination reaction with corresponding recombinase, upon which the backbone sequence of the donor vector II between the two S2 sites is removed and the ends of the acceptor vector bearing the inserted genes or gene groups are joined to form a circular plasmid;
(4) repeating said step (2) and step (3) with alternate donor vector plasmids containing target gene or gene group and the acceptor vector plasmid obtained in the former step, until all target genes or DNA fragments being linked into the acceptor vector to finish a designed vector construct.

6. An acceptor vector plasmid according to claim 1, which comprises all or part of the components shown in FIG. 1A or all or part of the DNA sequence SEQ ID NO: 1.

7. A donor vector I plasmid according to claim 1, which comprises all or part of the components shown in FIG. 1B or all or part of the DNA sequence SEQ ID NO: 2.

8. A donor vector II plasmid according to claim 1, which comprises all or part of the components shown in FIG. 1C or all or part of the DNA sequence SEQ ID NO: 3.

9. The application of the method of claim 1, wherein multiple genes or DNA fragments of interest are combined to a vector to create a desired vector construct, or the genes combined in the vector construct are transferred together into selected recipients to obtain multiple gene-products or express multi-gene-depended characters.


SEQUENCE LISTING (3 sequences)

SEQ ID NO: 1 - DNA, 118 bp, Artificial Sequence
  Synthesized fragment of MCS-loxP-I-SceI
ggatccaagcttgtcgacggccggccgcggccgcataacttcgtatagcatacattatacgaagttatgggccgcattaccctgttatccctaggccccaattaggcctacccactag

SEQ ID NO: 2 - DNA, 245 bp, Artificial Sequence
  Synthesized fragment of loxP-PI-SceI-MCS-I-SceI
gcgcgctcataacttcgtatagcatacattatacgaagttatcagatctttttggctaccttaagtgccatttcattacctctttctccgcacccgacatagatgttaagagagtcatatcgatgcatgcggccgctagctcgagctctagaattctgcaggtaccgcggatccatgggcccgggactagtcgacatgtacaagcttgtagggataacagggtaatccctaagatctcagcgcgc

SEQ ID NO: 3 - DNA, 245 bp, Artificial Sequence
  Synthesized fragment of loxP-I-SceI-MCS-PI-SceI
gcgcgctcataacttcgtatagcatacattatacgaagttatcagatcttagggattaccctgttatccctacaagcttgtacatgtcgactagtcccgggcccatggatccgcggtacctgcagaattctagagctcgagctagcggccgcatgcatcgatatgactctcttaacatctatgtcgggtgcggagaaagaggtaatgaaatggcacttaaggtagccaaaaagatctcagcgcgc